(19) United States
(12) Patent Application Publication
(10) Pub. No.: US 2004/0122283 A1
(43) Pub. Date: Jun. 24, 2004

(54) Cannula tip for a cardiac assist device

(75) Inventors: Yukihiko Nose, Houston, TX (US); Toshiyuki Shinohara, Kawasaki (JP); Kuniyoshi Watanabe, Houston, TX (US)

Correspondence Address: LAW OFFICE OF BARRY R LIPSITZ, 755 MAIN STREET, MONROE, CT, 06468, US

(73) Assignees: Miwatec Incorporated, Kanagawa-Ken (JP); Baylor College of Medicine, Houston, TX

(21) Appl. No.: 10/655,489

(22) Filed: Sep. 3, 2003

Related U.S. Application Data
(60) Provisional application No. 60/409,885, filed on Sep. 10, 2002.

(51) Int. Cl. A61N 1/362

(52) U.S. Cl. 600/16

(57) ABSTRACT

The present invention provides an inflow cannula tip that is shaped to prevent suction with the ventricular wall. In particular, the inlet portion of the cannula tip comprises two projections extending from the cylindrical body of the cannula tip with gaps between said projections. The projections may have beveled edges. The inlet portion of the cannula tip can accept blood flow from either the gaps in the side or from the bottom of the tip. Even if the edge of the cannula tip is close to or even touching ventricle wall, a suction condition will not result, as blood is allowed to flow through the gaps between the projections.

This application claims the benefit of U.S. provisional patent application No. 60/409,885 filed on Sep. 10, 2002, which is incorporated herein and made a part hereof by reference.

### BACKGROUND OF THE INVENTION

The present invention relates to the field of cardiac assist devices, such as blood pumps and the like. More specifically, the present invention relates to a cannula tip for use at the inflow of a blood pump that is designed to avoid the creation of a suction condition.

Bypass type cardiac assist devices are used frequently in advanced stages of heart failure to assist the failing human heart. It is typical for such devices to be used as left ventricular assist devices (LVAS) or right ventricular assist devices (RVAS).

Approximately 30% of the deaths of LVAS patients are the result of excessive venous pressure, increase in the hepatic portal vein pressure, and multi-organ failure. Over 60,000 patients a year who experience heart failure will require cardiac assist devices.

One problem associated with an implanted cardiac assist device is decreased pump flow volume which may result when the inflow cannula tip becomes attached to the ventricle wall resulting in a “suction condition”. In addition to decreasing the pump flow volume, such a suction condition will also limit the output of the natural heart, leading to sudden decrease in blood pressure, blood circulatory failure, and possible death.

The suction condition is more prevalent with a right ventricular assist pump. The inflow conduit of a right ventricular assist pump is inserted directly through the right ventricular wall near the pulmonary arterial valve. Anatomically there is not enough space available at this location between the right ventricular wall and the septum. Thus, a suction condition will occur easily unless a proper inflow tip is provided.

This suction condition may be caused by improper positioning of the cannula tip, excessive pump flow volume, outside pressure of the heart, etc. It is imperative to prevent the occurrence of such a suction condition.

It would be advantageous to provide a cannula tip for use at the pump inflow which is shaped to avoid the occurrence of the suction condition.

The inflow cannula tip of the present invention provides the foregoing and other advantages.

### SUMMARY OF THE INVENTION

The present invention relates to a cannula tip for the inflow of a blood pump that is designed to prevent the occurrence of a suction condition. The inventive inflow cannula tip is particularly useful for a right ventricular assist pump. The inventive inflow cannula tip may also be used with a left ventricular assist device.

In accordance with one example embodiment of the invention, an inflow cannula tip for use with a cardiac assist device is provided. The cannula tip has a hollow cylindrical body with a first end adapted for connection to a cannula. The cannula tip also has a hollow inlet portion at a second end of the body comprising at least two projections extending from the body and forming gaps in the side of the inlet portion between the projections.

In an example embodiment, the projections may comprise semi-circular projections. The gaps between the projections may be approximately U-shaped gaps. The projections may have beveled edges.

The cannula tip may be integrally formed with said cannula. Alternatively, the cannula tip may be formed as a separate piece that may be connected to the cannula.

The cannula tip may be made from a biocompatible hard plastic, such as polyvinyl or polyurethane. Alternatively, the cannula tip may be made from a titanium alloy.

The cannula tip may be adapted for use with a left ventricular assist device or a right ventricular assist device. Further, the cannula tip may be adapted for use with one of a pulsatile assist device or a non-pulsatile assist device.

### BRIEF DESCRIPTION OF THE DRAWINGS

The present invention will hereinafter be described in conjunction with the appended drawing figures, wherein like reference numerals denote like elements, and:

FIG. 1 (FIGS. 1A and 1B) shows the operation of a prior art cannula tip;

FIG. 2 shows a perspective view of an example embodiment of the inventive cannula tip;

FIG. 3 shows a further perspective view of an example embodiment of the inventive cannula tip; and

FIG. 4 (FIGS. 4A and 4B) shows the operation of an example embodiment of the inventive cannula tip.

### DETAILED DESCRIPTION

The ensuing detailed description provides exemplary embodiments only, and is not intended to limit the scope, applicability, or configuration of the invention. Rather, the ensuing detailed description of the exemplary embodiments will provide those skilled in the art with an enabling description for implementing an embodiment of the invention. It should be understood that various changes may be made in the function and arrangement of elements without departing from the spirit and scope of the invention as set forth in the appended claims.

The present invention relates to a cannula tip for the inflow of a blood pump that is designed to prevent the occurrence of a suction condition. This particular design of an inflow cannula tip is particularly useful for a right ventricular assist pump. The inventive inflow cannula tip may also be used with a left ventricular assist device.

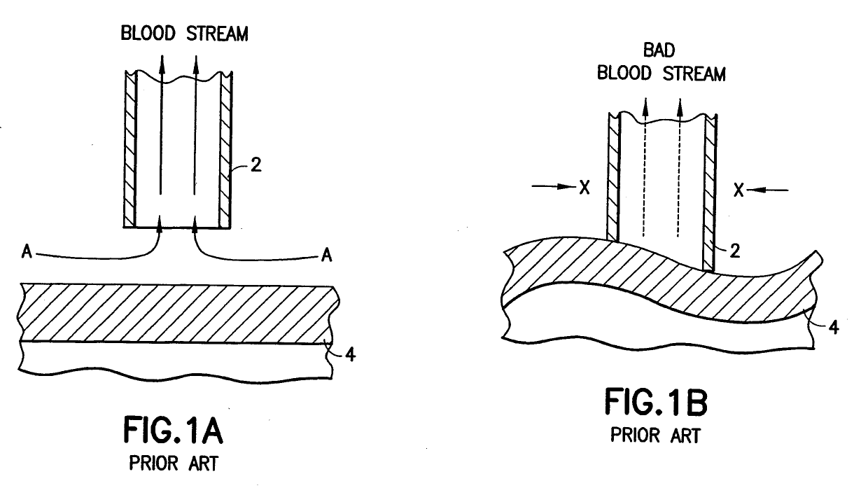

FIGS. 1A and 1B show a prior art cannula tip 2 in operation. This prior art tip 2 is in the form of a cylindrical tube. FIG. 1A shows the prior art cannula tip 2 in normal operation with blood flow in the direction of arrows A. However, the cylindrical tube design of the prior art cannula tip 2 leads to a suction condition with the ventricular wall 4 as shown in FIG. 1B. Such a suction condition hinders blood flow. This suction condition may be caused by improper positioning of the cannula tip, excessive pump flow volume, outside pressure of the heart, or a combination thereof.

With such a cylindrical structure of the prior art cannula tip 2, it is difficult to recover from the suction condition. Attempts have been made to solve this problem in the prior art. For example, prior art cannula tips having a cylindrical structure may be provided with multiple holes in the side of the cylinder. Such a design acts to prevent the suction condition. However, such the holes of such a prior art design have a tendency to become occluded by blood clots during long-term implantation. The cannula tip of the present invention not only prevents the suction condition, but also prevents the formulation of blood clots.

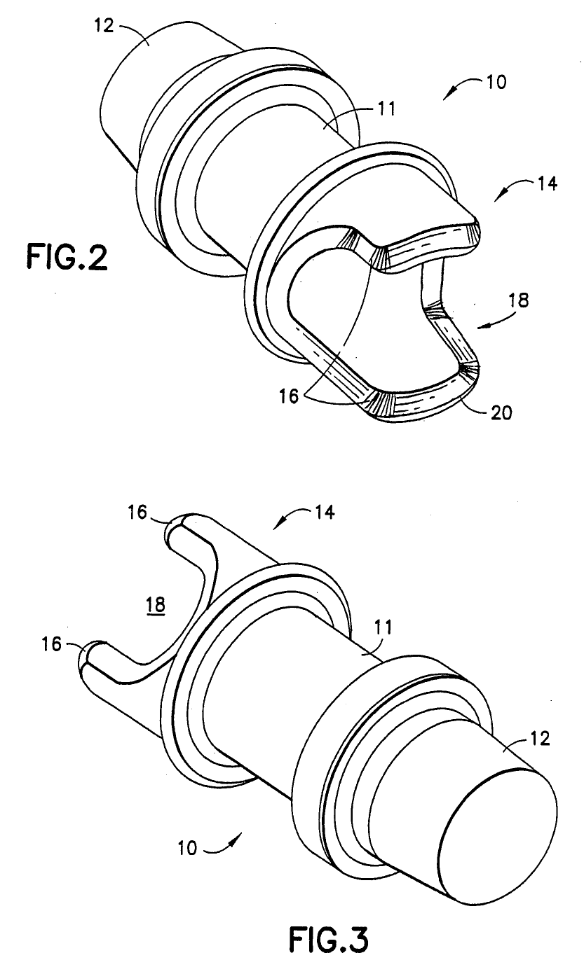

The mouth (inlet) of the cannula tip of the present invention is shaped to prevent suction when in contact with the ventricular wall. Perspective views of an example embodiment of the present invention are shown in FIGS. 2 and 3. The inventive cannula tip 10 has a hollow cylindrical body 11 with a first end 12 adapted for connection to a cannula (not shown). A hollow inlet portion 14 of the cannula tip 10 may comprise two projections 16 extending from the cylindrical body 11 of the cannula tip 10 which projections 16 are positioned so as to provide gaps 18 in the side of the inlet portion 14.

The projections 16 may be in the form of semi-circular projections. The gaps 18 may be generally U-shaped gaps in the inlet portion 14 of the cannula tip 10. The projections 16 may have beveled edges 20.

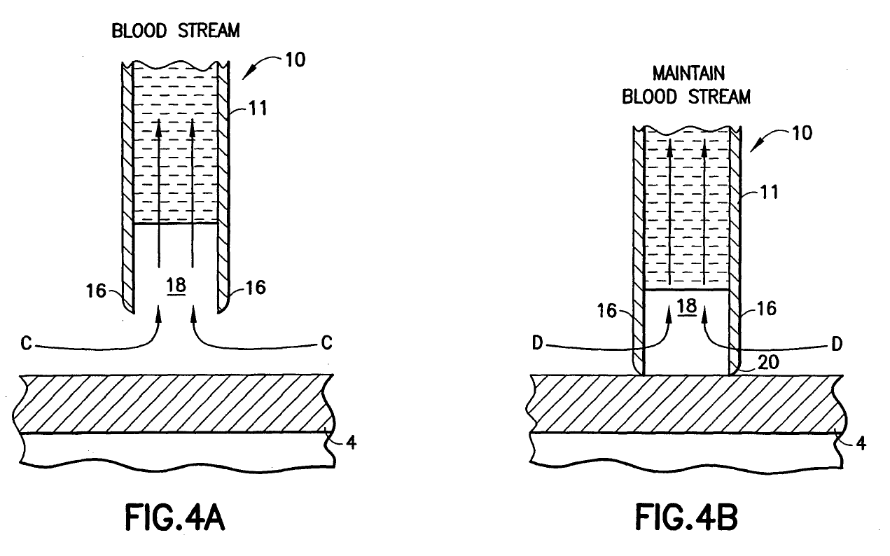

The inlet portion 14 of the tip 10 can accept blood flow from either the side or the bottom of the cannula tip as shown in FIGS. 4A and 4B. FIG. 4A shows the operation of the cannula tip 10 of the present invention in a normal operating condition (i.e., not in contact with the ventricle wall 4). In FIG. 4A, blood flow may enter the cannula tip 10 from the bottom as shown by arrows C. As shown in FIG. 4B, if the projections 16 of the cannula tip 10 of the present invention should come in contact with the ventricle wall 4, no suction condition will result, since blood flow may still enter the inlet portion of the cannula tip 10 through the gaps 18 between the projections 16, as shown by arrows D.

Those skilled in the art will appreciate that, although the figures show two projections 16 extending from the cannula tip body 11, more than two projections extending from the body 11 of the cannula tip 10 may be provided having gaps 18 therebetween. Also, it should be appreciated that the cannula tip 10 may comprise various other shapes which will similarly prevent a suction condition by allowing blood flow even when the tip is close to or touching the ventricular wall.

The inflow cannula tip 10 can be made from a biocompatible hard plastic (e.g., polyvinyl or polyurethane) for short-term use or from titanium alloy for long term use. This tip 10 may be a separate piece that can be connected to a cannula. Alternatively, the tip 10 may be formed together with a cannula as a single piece.

The cannula tip 10 of the present invention does not require complex structure and design. Further, the inventive tip 10 does not interfere with the insertion of the cannula into the ventricle.

The cannula tip 10 of the present invention can be used not only with a Right Ventricular Assist Device but also a Left Ventricular Assist Device. Further, the inventive cannula tip 10 can be used for both pulsatile heart assist devices and non-pulsatile heart assist devices. The cannula tip 10 may also be successfully used in other applications where the insertion of a cannula is required.

It should now be appreciated that the present invention provides an advantageous inflow cannula tip that prevents the formation of a suction condition.

Although the invention has been described in connection with various illustrated embodiments, numerous modifications and adaptations may be made thereto without departing from the spirit and scope of the invention as set forth in the claims.

What is claimed is:

1. An inflow cannula tip for use with a cardiac assist device, comprising:
a hollow cylindrical body having a first end adapted for connection to a cannula; and
a hollow inlet portion at a second end of said body comprising at least two projections extending from said body and forming gaps in the side of the inlet portion between said projections.

2. An inflow cannula tip in accordance with claim 1, wherein said projections comprise semi-circular projections.

3. An inflow cannula tip in accordance with claim 1, wherein said gaps are approximately U-shaped gaps.

4. An inflow cannula tip in accordance with claim 1, wherein said projections have beveled edges.

5. An inflow cannula tip in accordance with claim 1, wherein said cannula tip is integrally formed with said cannula.

6. An inflow cannula tip in accordance with claim 1, wherein said cannula tip comprises biocompatible hard plastic.

7. An inflow cannula tip in accordance with claim 6, wherein said cannula tip comprises one of polyvinyl or polyurethane.

8. An inflow cannula tip in accordance with claim 1, wherein said cannula tip comprises a titanium alloy.

9. An inflow cannula tip in accordance with claim 1, wherein said cannula tip is adapted for use with one of a left ventricular assist device or a right ventricular assist device.

10. An inflow cannula tip in accordance with claim 1, wherein said cannula tip is adapted for use with one of a pulsatile assist device or a non-pulsatile assist device.
